(19) United States
(12) Patent Application Publication
(10) Pub. No.: US 2021/0001145 A1
(43) Pub. Date: Jan. 7, 2021

(54) CONTROLLING MYOPIA IN HUMANS

(71) Applicant: Sustainable Eye Health IP PTY Ltd, Sydney (AU)

(72) Inventor: Stephen Mason, Surry Hills (AU)

(21) Appl. No.: 16/976,658

(22) PCT Filed: Feb. 28, 2019
(86) PCT No.: PCT/AU2019/050173

§ 371 Date: Aug. 28, 2020

(30) Foreign Application Priority Data
Feb. 28, 2018 (AU) 2018900651
Apr. 26, 2018 (AU) 2018901382

(51) Int. Cl. A61N 5/06; H05B 45/20; H05B 45/10

(52) U.S. Cl. CPC A61N 5/0613; H05B 45/20; A61N 2005/0663; A61N 2005/0632; A61N 2005/0652; H05B 45/10

(57) ABSTRACT

The present invention is broadly directed to an artificial lighting system 30 for emitting artificial light in an indoor environment for controlling myopia in humans. The artificial lighting system 30 generally comprises:
1. one or more luminaires such as 34a to 34d designed to directly generate and emit artificial light without filters which substantially simulates the effect of sunlight and is of a predetermined wavelength emission spectrum (a) higher in its proportion of wavelengths at or around 480 nm relatively to neighbouring wavelengths, and (b) lower in its proportion of high energy visible light;
2. an electronic control module 36 operatively coupled to the luminaires such as 34a to control their emission of the artificial light.

### TECHNICAL FIELD

The present invention relates broadly to an artificial light source for controlling myopia in humans and relates particularly, although not exclusively, to an artificial lighting system and method for emitting artificial light suitable for controlling myopia in humans.

### BACKGROUND OF INVENTION

Myopia or short-sightedness is reaching epidemic levels where for example it is estimated that half of young adults in the US and Europe and up to 90% of teenagers and young adults in China are myopic. It is also estimated that by 2020 around one third of the world's population could be short-sighted and by 2050 half of the world's population will be short-sighted. Of these, 10% are likely to be highly myopic (more than −5.00 Dioptres) leading to a high risk of ocular morbidity in older age. This is destined to place pressure on health costs for both developed and developing nations globally. It is understood that myopia is most commonly a consequence of an axial elongation of the eyeball such that light from far objects is focused in front of the retina, rather than directly on it. The various approaches to arresting or controlling the progression of myopia include:
i) optical devices such as multifocal spectacle lenses or contact lenses including dual-focus and multi-focal contact lenses;
ii) atropine or atropine-like parasympathomimetic eye-drops with a neurotransmitter-blocking agent to paralyse accommodation of the ciliary muscle of the eye which has the undesirable effect of dilating the pupil and causing blurred vision and glare sensitivity with these pharmacological agents also being considered to provide a dopamine-agonist effect at the retinal level, and the effectiveness of this therapy being questionable as research shows myopia rapidly increases after therapy is stopped;
iii) optical correction including laser refractive surgery to restore visual acuity with the attendant risks of eye and vision damage as a consequence of surgery and disruption of the surface of the eye such risk being inaccurate ablation of the anterior optical surface of the eye or infection with disruption of the corneal epithelium being a barrier to infection by bacteria and or other microbes, and furthermore laser refractive surgery is not typically an option for high-myopia for risk of rendering the cornea adversely thin post-operatively leading to catastrophic corneal ectasia and loss of vision;
iv) rigid contact lenses worn at night to flatten the cornea and cause it to become an oblate form known as orthokeratology which are not suitable for all forms of ametropia typically those forms of myopia that also have an element of astigmatism in addition to the myopia or when the myopia is high.

More recently it has been found by repeated research studies by various investigators particularly in children that increased day-time outdoor light exposure associates with a reduced prevalence of myopia. It has also been suggested that increased night-time exposure to very low levels of artificial light (night-lights) may be associated with increased myopia development in the very young. Therefore, in order to avoid eye growth in childhood the peer-reviewed academic literature teaches increased natural light exposure to children by increasing their day-time activity outdoors, typically a minimum of two to three hours to reduce the risk of incidence and progression of myopia.

### SUMMARY OF INVENTION

According to a first aspect of the present invention there is provided an artificial lighting system for emitting artificial light for controlling myopia in humans, said lighting system comprising:
(A) an artificial light source of a light-emitting diode type, said light source designed to directly generate and emit artificial light without filters:
   i) at a predetermined wavelength emission spectrum substantially simulating the effect of sunlight, said wavelength spectrum being within a range of wavelengths detectable by an individual's eyes at a retinal level;
   ii) at a predetermined level of illuminance measured at the eyes of at least 300 lux and up to around 1,000 lux measured at or around desk height;
   iii) the predetermined wavelength emission spectrum being a) higher in its proportion of wavelengths at 480 nm±5 nm relative to neighbouring wavelengths, and b) relatively lower in its proportion of high energy visible light of wavelengths at between 415 nm to 455 nm,
(B) an electronic control module operatively coupled to the artificial light source to control its emission of the artificial light which when exposed to an individual's eyes for a predetermined exposure period of time of at least 180 minutes on average per day and at least 1,800 lux-hours per day is sufficient to trigger a neurological response in the retina of each of the eyes which is effective in contributing to a reduction in the onset or progression of myopia in the individual's eyes.

Preferably the electronic control module includes a built-in control board. More preferably the electronic control module is configured to vary one or more characteristics of the artificial light at predetermined time intervals. Even more preferably the electronic control module is configured to vary one or more of the artificial light characteristics including spectral power distribution, wavelength emission spectrum, correlated colour temperature (CCT), level of illuminance or luminance, exposure period of time, and periodicity of these characteristics. Still more preferably the electronic control module is configured to automatically control said one or more artificial light characteristics.

Preferably the artificial lighting system also comprises a sensor arranged to detect ambient light and operatively coupled to the electronic control module to modulate one or more characteristics of the artificial light. More preferably the sensor is configured to communicate with the electronic control module in providing feedback to said control module to adjust at least the level of illuminance of the artificial light depending on the level of ambient light detected by the sensor.

Preferably the artificial light source of the light-emitting diode type comprises a plurality of semiconductor layers each inherently designed to generate light at respective of a range of wavelength emission spectrums, said semiconductor layers arranged relative to one another wherein the light generated from each of said layers combines to directly generate and emit the artificial light at the predetermined wavelength emission spectrum and level of illuminance. More preferably the plurality of semiconductor layers is in the form of a grid of light emitting diodes each inherently designed to generate light at respective of distinct wavelength or colour spectrums corresponding to the range of wavelength emission spectrums. Even more preferably the grid of light emitting diodes combine to directly generate and emit said artificial light.

According to a second aspect of the invention there is provided an artificial light source for emitting artificial light for controlling myopia in humans, said light source being a light-emitting diode type designed to directly generate and emit artificial light without filters:
i) at a predetermined wavelength emission spectrum substantially simulating the effect of sunlight, said wavelength spectrum being within a range of wavelengths detectable by an individual's eyes at a retinal level;
ii) at a predetermined level of illuminance measured at the eyes of at least 300 lux and up to around 1,000 lux measured at or around desk height;
iii) the predetermined wavelength emission spectrum being a) higher in its proportion of wavelengths at 480 nm±5 nm relative to neighbouring wavelengths, and b) relatively lower in its proportion of high energy visible light of wavelengths at between 415 nm to 455 nm,
   said artificial light when exposed to an individual's eyes for a predetermined exposure period of time of at least 180 minutes on average per day and at least 1,800 lux-hours per day being sufficient to trigger a neurological response in the retina of each of the eyes which is effective in contributing to a reduction in the onset or progression of myopia in the individual's eyes.

Preferably the artificial light source of the light-emitting diode type comprises a plurality of semiconductor layers each designed to generate light at respective of a range of wavelength emission spectrums, said semiconductor layers arranged relative to one another wherein the light generated from each of said layers combines to directly generate and emit the artificial light at the predetermined wavelength emission spectrum and level of illuminance. More preferably the plurality of semiconductor layers is in the form of a grid of light emitting diodes each inherently designed to generate and emit light at respective of distinct wavelength or colour spectrums corresponding to the range of wavelength emission spectrums. Even more preferably the grid of light emitting diodes combine to directly generate and emit said artificial light.

According to a third aspect of the invention there is provided a method of controlling myopia in humans, said method comprising the steps of:
(A) directly generating and emitting artificial light from an artificial light source without filters, said artificial light being emitted:
   i) at a predetermined wavelength emission spectrum substantially simulating the effect of sunlight, said wavelength spectrum being within a range of wavelengths detectable by an individual's eyes at a retinal level;
   ii) at a predetermined level of illuminance measured at the eyes of at least 300 lux and no more than around 1,000 lux measured at or around desk height;
   iii) the predetermined wavelength emission spectrum being a) higher in its proportion of wavelengths at 480 nm±5 nm relative to neighbouring wavelengths, and b) relatively lower in its proportion of high energy visible light of wavelengths at between 415 nm to 455 nm,
(B) exposing an individual's eyes to the artificial light for a predetermined exposure period of time of at least 180 minutes on average per day and at least 1,800 lux-hours per day, said artificial light at said predetermined exposure being sufficient to trigger a neurological response in the retina of each of the eyes which is effective in contributing to a reduction in the onset or progression of myopia in the individual's eyes.

It is understood by the applicant that the predetermined wavelength emission spectrum, also referred to in the literature as the spectral power distribution, can be varied with the time of day so that the retinal irradiance levels from the light source as well as wavelengths of illumination are strategically controlled so that the sum of retinal illuminance over time along with spectral power distribution are consistent with and respect the natural diurnal cycle, typically referred to as the circadian rhythm, known to be positively beneficial for health and well-being. This time of day variation in the spectral power distribution may be effected by software connection via electronic controllers to an LED or other light sources.

Preferably the step of exposing the individual's eyes to artificial light involves varying one or more characteristics of the artificial light at predetermined time intervals within the predetermined exposure period. More preferably said one or more characteristics of the artificial light include spectral power distribution, wavelength emission spectrum, CCT, level of illuminance or luminance, exposure period of time, and periodicity of these characteristics.

Preferably the step of exposing the individual's eyes to artificial light involves substantially continuous exposure to said artificial light at ambient levels of illuminance which substantially simulates sunlight in its chromatic range of wavelengths.

It is understood by the applicant that exposure of an individual's eyes to artificial light at or around this wavelength of 480 nm triggers a neurological response at a local level in the retina of each of the eyes which is effective in contributing to a reduction in the onset or progression of myopia in the individual's eyes. It is also understood by the applicant that the wavelength at or around 480 nm is the wavelength of light absorbed substantially by melanopsin within intrinsically sensitive retinal ganglion cells that initiates the cascade of neurological responses at a retinal level for maintenance of the emmetropisation process and stability of the underlying anatomy of the eyes and so leading to a reduced risk of development of axial elongation of the eyes typically referred to as myopia.

According to a fourth aspect of the invention there is provided use of an artificial light source in the manufacture of an artificial lighting system for controlling myopia in humans.

### BRIEF DESCRIPTION OF DRAWINGS

In order to achieve a better understanding of the nature of the present invention a preferred embodiment of a method of controlling myopia in humans together with an artificial light source for emitting artificial light suitable for controlling myopia will now be described, by way of example only, with reference to the accompanying drawings in which:

FIG. 1 is a flowchart illustrating the general steps involved in a method of controlling myopia according to a preferred embodiment of one aspect of the invention;

FIGS. 2A and 2B are comparative graphs for different artificial light sources illustrating a predetermined wavelength emission spectrum for artificial light used in the preferred methodology of controlling myopia;

FIG. 3 is a schematic illustration of an artificial lighting system for emitting artificial light in an indoor environment for controlling myopia in humans, the system being in accordance with a preferred embodiment of another aspect of the invention;

FIG. 4 is a schematic illustration of a luminaire including an artificial light source for controlling myopia in humans, the light source being in accordance with a preferred embodiment of a further aspect of the invention;

FIGS. 5A and 5B are schematic illustrations in perspective and sectional views respectively of alternative embodiments of an artificial light source of a light emitting diode type according to the invention.

### DETAILED DESCRIPTION

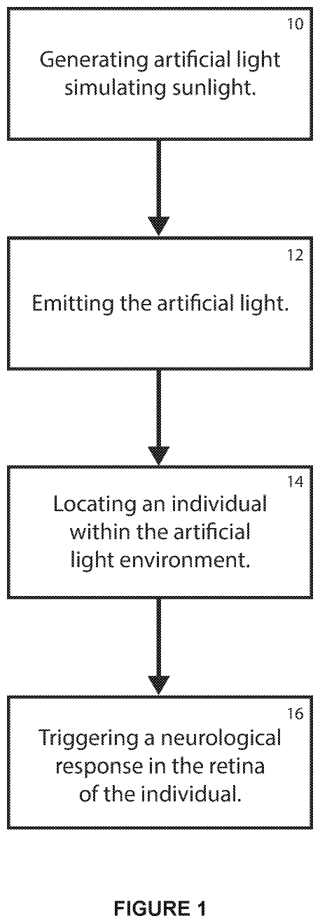

In the flowchart of FIG. 1 the general steps involved in exposing an individual's eyes to artificial light to control myopia include:
1. generating the artificial light which substantially simulates the effect on the eyes of sunlight at 10;
2. emitting the artificial light at a predetermined level of illuminance for a predetermined exposure period of time at 12;
3. locating the individual's eyes within the environment in which the artificial light is emitted at 14 for the predetermined exposure period;
4. triggering a neurological response at a local level in the retina of each of the eyes which is effective in contributing to a reduction in the onset or progression of myopia in the individual's eyes at 16.

In this embodiment the individual's eyes are exposed to the artificial light which is emitted within an indoor environment occupied by the individual. The indoor environment may be in the form of a classroom occupied by children and the artificial light is emitted from an artificial light source located within the classroom or other building occupied by the children.

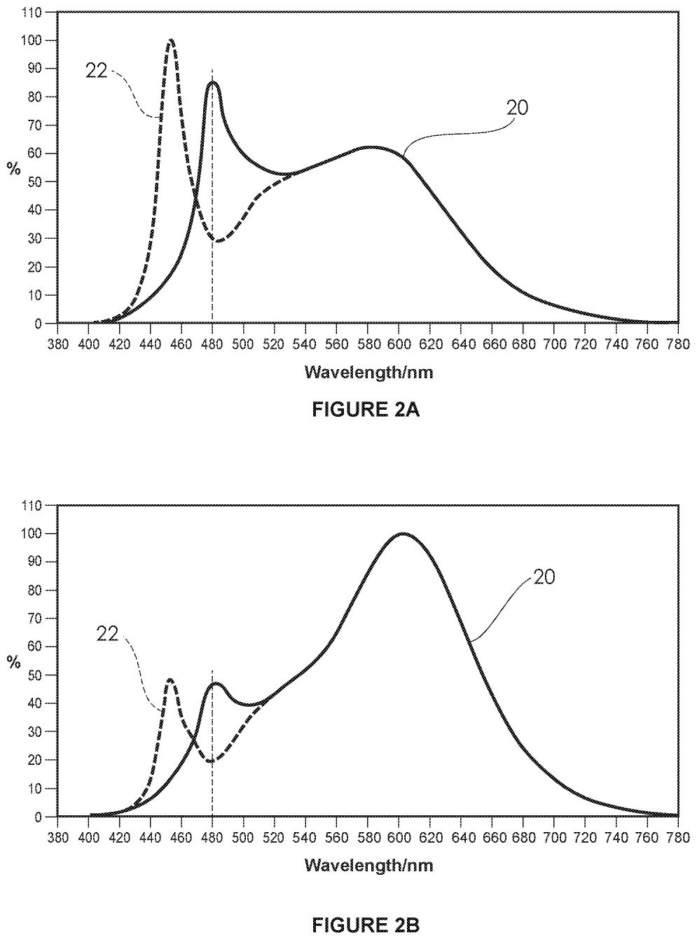

In this embodiment the subject's eyes are exposed to the artificial light at a predetermined wavelength emission spectrum. The predetermined wavelength emission spectrum is within a range of wavelengths detectable by an individual's eyes at a retinal level. FIGS. 2A and 2B illustrate exemplary wavelength emission spectrums 20 for deployment in the preferred method of controlling myopia. The graphs correspond to different artificial light sources of an LED type. The preferred spectrum 20 for each of the LEDs is shown in solid line detail whereas a conventional spectrum 22 for the comparable LED is shown in broken line detail. It can be seen that the wavelength emission spectrum 20 substantially simulates the effect of sunlight, and the comparable LED, with the following exceptions:
1. the wavelength emission spectrum 20 is higher in its proportion of wavelengths at or around 480 nm relative to neighbouring wavelengths;
2. the wavelength emission spectrum 20 is relatively lower in its proportion of high energy visible light of wavelengths at between 415 nm to 455 nm.

This modification of indoor lighting for ameliorating the negative impact of inadequate retinal luminance also recognises the importance of protecting the retina particularly the central retina anatomically referred to as the macular from undesirable and potentially damaging radiation in the range of 415 nm to 455 nm which is a threat to the long-term health and integrity of the underlying retinal pigmentary epithelium upon which the retina sits as well as the mRNA which is central to normal cellular metabolism. Long term exposure to aforesaid range of light 415 nm to 455 nm referred to as ‘high energy visible’ light being absorbed by the retinal pigmentary epithelium is a risk to the development of macular degeneration, a scarring effect suffered by the macular causative of the most common form of legal blindness in developed nations typically referred to as macular degeneration.

In this embodiment the individual's eyes are exposed to artificial light at a level of illuminance of at least 300 lux and typically around 3000 lux measured at desk height for an exposure period of around two hours on average per day. It should be understood that the required level of illuminance at the individual's eyes may require higher levels of ambient illuminance, for example anywhere from 2000 to 6000 lux measured at either desk height or at the distance of the subjects eyes from the luminaire. In any event the level of illuminance and exposure period with the weighting of radiation at or around 480 nm is sufficient to trigger the required neurological response in the retina of at least one of the eyes which is exposed to the artificial light supporting the emmetropisation process and normalisation of the anatomy of the eyeball. It is understood by the applicant that this trigger of the neurological response at a local level in the retina when exposed to artificial light having at least the wavelength and illuminance characteristics of the present invention is effected by i) stimulating intrinsically photosensitive retinal ganglion cells (ipRGC's), ii) said stimulated ipRGC's in turn triggering amacrine cells which reside in the inner nuclear layer of the retina. The amacrine cells when triggered release dopamine and or other neuro-transmitters that begins the neurological response of the visuo-sensory system essential for maintenance of the emmetropisation process in children and adults thereby controlling myopia. Unlike prior art disclosures relating to the preservation of circadian rhythm which is controlled in the mid-brain, the present invention thus controls myopia by neurological triggers at a local level within the eyes.

The artificial light exposure at the relatively high level of illuminance simulating the effect of sunlight on the eyes may be provided continuously or intermittently. In either case the daily level of exposure to elevated illuminance averaged at least to 2000 lux over a period corresponding to the length of an average day of twelve hours should be at least two hours to trigger the adequate ipRGC response. In practice, the artificial light source may be configured to provide around 600 lux for around 3 hours of a morning and around 400 lux for around 2.5 hours of an afternoon. This translates to 2800 lux-hours per day or around 14000 lux-hours for a five (5) day period.

In this embodiment exposure of the individual's eyes to artificial light is at ambient levels of illuminance which substantially simulate sunlight in its chromatic range of wavelengths and is typically in the range of 400 nm to 720 nm being the range of wavelengths to which the human eye can detect at the retinal level. This means that the simulated sunlight of the artificial light is characterised by a correlated colour temperature (CCT) of between 200K to 6000K. The CCT of the artificial light may be varied at predetermined time intervals within the exposure period where the CCT is selected to substantially preserve circadian rhythm in the individual. The artificial light may also at these predetermined time intervals be varied in its other characterising features including but not limited its wavelength emission spectrum, level of illuminance, and exposure period of time including intermittent exposure at a predetermined frequency.

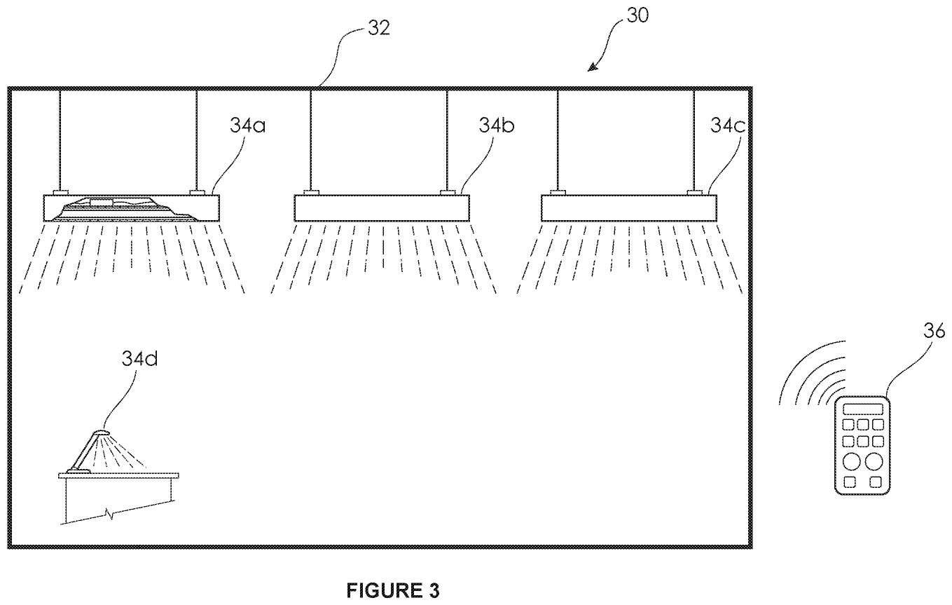

FIG. 3 illustrates a preferred embodiment of an artificial lighting system 30 according to another aspect of the invention for emitting artificial light in an indoor environment for controlling myopia in humans. The lighting system 30 is designed to emit artificial light in an indoor space of a building such as but not limited to a classroom, office, or lecture room 32.

The artificial lighting system 30 of this embodiment broadly comprises:
1. one or more luminaires such as 34a to 34d designed to directly generate and emit artificial light without filters which substantially simulates the effect of sunlight and is of a predetermined wavelength emission spectrum a) higher in its proportion of wavelengths at or around 480 nm relative to neighbouring wavelengths, and b) lower in its proportion of high energy visible light;
2. an electronic control module 36 operatively coupled to the luminaires such as 34a to control their emission of the artificial light.

Typically the luminaire itself includes a built-in control board which functions as the electronic control module. Alternatively and as illustrated, the electronic control module 36 may wirelessly communicate with the luminaires such as 34a and may be local to or remote from the building 32. Communication of the control module 36 with the luminaires 34a may alternatively be by Ethernet. The control module 36 may be in the form of a computer operated by software or an appropriate app, typically loaded on a tablet or other mobile device. In any of these configurations, the control module 36 is designed to vary one or more characteristics of the artificial light at predetermined time intervals. In line with the preferred method of controlling myopia, the artificial light may be varied in terms of its wavelength emission spectrum, CCT, level of illuminance, and/or exposure period of time. The emission of artificial light under control of the electronic control module 30 may be effected:
1. manually where for example a teacher at their discretion adjusts the CCT and/or luminance of the artificial light to influence behaviour of children or other individuals exposed to the artificial light; and/or
2. automatically in accordance with a predetermined algorithm or program which may for example adjust the CCT and/or luminance of the artificial light to substantially preserve circadian rhythm.

In adjusting or controlling characteristics of the artificial light, the key consideration is to maintain exposure within the indoor environment at a level of illuminance and period of time which is sufficient to trigger the required neurological response at a local level in the retina in at least one of the eyes. The applicant understands that this required level of exposure and subsequent neurological response is effective in contributing to a reduction in the onset or progression of myopia and inhibition of the risk of macular degeneration.

In this embodiment the luminaires such as 34a to 34d are mounted to a ceiling of the classroom 32 or associated with a desktop within the classroom 32. It is expected that the relative proximity of the luminaire to the individual's eyes will influence the level of illuminance of the luminaire itself in order to provide sufficient ambient illuminance at the eyes and to the retina to trigger the required neurological response. The overhead luminaires such as 34a to 34c may be designed with an ambient level of illuminance of around 1000 lux measured at or around desk height whereas the desktop luminaires such as 34d may emit artificial light at ambient levels of around 600 lux or less. The luminaires 34a to 34c are each designed with an ambient level of illuminance (or sufficient brightness) to trigger the intrinsically photosensitive retinal ganglion cells (ipRGC's), typically of at least 300 lux measured at desk height when ceiling mounted. The desktop luminaires 34d being at a closer proximity to the eyes are designed to enhance the emission spectrum at wavelengths at or around 480 nm to promote emmetropisation but limiting the wavelength range of 415 nm to 455 nm to guard against the risk of macular degeneration.

The luminaire such as 34a may be equipped with a sensor (not shown) that allows modulation of the variables (lux, chromaticity and timing of changes) based on ambient light. The sensor is operatively coupled to the electronic control module and operates to ensure that illumination within the indoor environment is optimal for myopia control and accordingly governs the light output of the associated luminaires. The sensor may also provide feedback to the electronic control module or controller/software in order that the average lux exposure is observed so that the sum of the lux output for a period, typically a day, is adequate to provide for myopia control which has been shown to be a minimum of an average of 2000 lux-hours per day. The sensor may detect a sunny indoor environment to influence the control module in reducing the required illumination of the luminaires such as 36a and the reverse applies if illumination from sunlight is reduced.

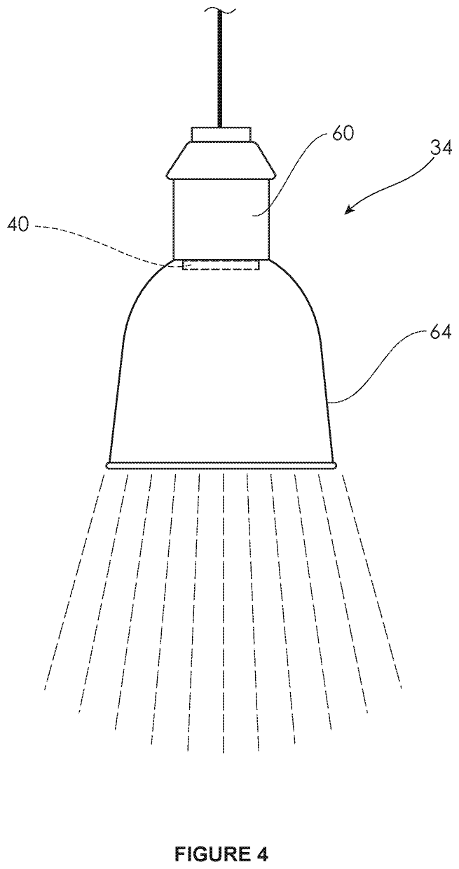

FIG. 4 is a schematic illustration of a luminaire 34 including an artificial light source 40 of one embodiment taken from the artificial lighting system 30 of FIG. 3. The artificial light source 40 of this particular embodiment is designed to emit artificial light:
1. having a wavelength emission spectrum
   i) higher in its proportion of wavelengths at or around 480 nm relative to neighbouring wavelengths; and
   ii) being relatively lower in its proportion of high energy visible light of wavelengths at between around 415 nm to 455 nm;
2. at ambient levels of illuminance of around 1000 lux measured at desk height.

This exemplary wavelength emission spectrum is illustrated in FIGS. 2A and 2B and it is understood by the applicant that artificial light characterised in this way, and in particular higher in its proportion of wavelengths at or around 480 nm relative to neighbouring wavelengths, is effective in triggering the required neurological response at a local level in the retina. This favourable wavelength stimulates intrinsically photosensitive ganglion cells (ipRGCs) which leads to the release of dopamine from amacrine cells within the retina which is understood to inhibit the development and progression of myopia.

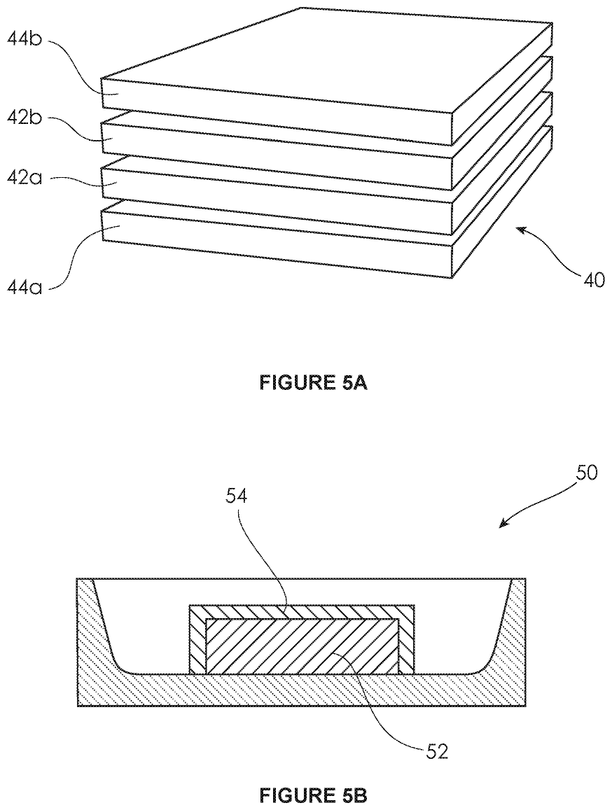

As schematically illustrated in FIGS. 5A and 5B, the artificial light sources 40 and 50 of these embodiments are of a light-emitting diode type (LED). The LED source 40 of FIG. 5A includes a plurality of semiconductor layers such as 42a and 42b of an electroluminescent material inherently designed to directly generate and emit artificial light of the predetermined wavelength emission spectrum when excited by electrons. In a conventional manner the semiconductor layers 42a/b are excited by electrons injected by electrical current into the layers 42a/b. Each of the semiconductor layers such as 42a/b generates light at their respective and fixed wavelength spectrum and together the layers 42a/b combine to directly generate the artificial light without filters at the predetermined wavelength emission spectrum. The LED source 40 includes electrodes such as 44a and 44b sandwiched either side of the semiconductor layers 42a/b (and possibly other functional layers) for connection to a source of the electrical current. The LED source 40 itself is thus inherently designed and engineered for direct generation of the artificial light at the predetermined wavelength emission spectrum. In this embodiment the individual's eyes are exposed to the artificial light at the generated predetermined wavelength emission spectrum without any filters or other intermediate barriers for influencing the wavelength emission spectrum of the artificial light.

In an alternative embodiment, the artificial light source 50 of FIG. 5B is a LED type including a single semiconductor 52 of an electroluminescent material such as Gallium Nitride (GaN), or a derivative thereof, inherently designed to generate substantially blue light. The LED source 50 includes a phosphor layer 54 deposited across or covering the semiconductor 52. The phosphor layer 54 functions to modify the blue light of the semiconductor 52 wherein the LED source 50 directly generates and emits a “white light” having the predetermined wavelength emission spectrum, limiting the proportion of potentially damaging high energy visible light.

The LED source such as 40 is designed to substantially mimic sunlight emitting a range of wavelengths of around but not limited to 400 nm to 720 nm at the preferred wavelength emission spectrum being i) higher in its proportion of wavelengths at or around 480 nm relative to neighbouring wavelengths, and ii) lower in its proportion of high energy visible light. The light source 40 of this embodiment is incorporated in a luminaire such as 34 which is otherwise of a conventional construction including an electrical assembly 42 connected to a source coupling 44, and a reflective housing or hood 46.

Now that a preferred embodiment of the invention has been described it will be understood that the method of controlling myopia and the related artificial lighting system have at least the following advantages:
1. the lighting system and method provide effective exposure to artificial light for reducing the onset or progression of myopia whilst minimising what otherwise would be damaging exposure to natural sunlight which can cause adverse effects such as skin cancer and retinal light damage with over-exposure;
2. the lighting system can be integrated or retrofitted with relative ease to an existing building without significant changes to existing infrastructure and without requiring any behavioural changes in the individuals occupying the building;
3. the lighting system provides effective control of the key artificial light characteristics in order to effectively manage the control of myopia in an indoor environment;
4. the method and system lend themselves to controlling other artificial light characteristics influencing the effect of individuals exposed to that artificial light having benefit for productivity, learning, restlessness and mood.

Those skilled in the art will appreciate that the invention described herein is susceptible to variations and modifications other than those specifically described. For example, the levels of illuminance and exposure periods disclosed may vary provided the necessary effect in triggering the required neurological response at a local level in the retina is achieved. The wavelength emission spectrum of the artificial light may also vary from that disclosed provided it is nonetheless higher in its proportion of wavelengths at or around 480 nm relative to neighbouring wavelengths, and lower in its proportion of high energy visible light. All such variations and modifications are to be considered within the scope of the present invention the nature of which is to be determined from the foregoing description.

1. An artificial lighting system for emitting artificial light for controlling myopia in humans, said lighting system comprising:
(A) an artificial light source of a light-emitting diode type, said light source designed to directly generate and emit artificial light without filters:
i) at a predetermined wavelength emission spectrum substantially simulating the effect of sunlight, said wavelength spectrum being within a range of wavelengths detectable by an individual's eyes at a retinal level;
ii) at a predetermined level of illuminance measured at the eyes of at least 300 lux and up to around 1,000 lux measured at or around desk height;
iii) the predetermined wavelength emission spectrum being a) higher in its proportion of wavelengths at 480 nm±5 nm relative to neighbouring wavelengths, and b) relatively lower in its proportion of high energy visible light of wavelengths at between 415 nm to 455 nm,
(B) an electronic control module operatively coupled to the artificial light source to control its emission of the artificial light which when exposed to an individual's eyes for a predetermined exposure period of time of at least 180 minutes on average per day and at least 1,800 lux-hours per day is sufficient to trigger a neurological response in the retina of each of the eyes which is effective in contributing to a reduction in the onset or progression of myopia in the individual's eyes.

2. An artificial lighting system as claimed in claim 1 wherein the electronic control module includes a built-in control board.

3. An artificial lighting system as claimed in claim 1 wherein the electronic control module is configured to vary one or more characteristics of the artificial light at predetermined time intervals.

4. An artificial lighting system as claimed in claim 3 wherein the electronic control module is configured to vary one or more of the artificial light characteristics including spectral power distribution, wavelength emission spectrum, correlated colour temperature (CCT), level of illuminance or luminance, exposure period of time, and periodicity of these characteristics.

5. An artificial lighting system as claimed in claim 4 wherein the electronic control module is configured to automatically control said one or more artificial light characteristics.

6. An artificial lighting system as claimed in claim 1 also comprising a sensor arranged to detect ambient light and operatively coupled to the electronic control module to modulate one or more characteristics of the artificial light.

7. An artificial lighting system as claimed in claim 6 wherein the sensor is configured to communicate with the electronic control module in providing feedback to said control module to adjust at least the level of illuminance of the artificial light depending on the level of ambient light detected by the sensor.

8. An artificial lighting system as claimed in claim 1 also wherein the artificial light source of the light-emitting diode type comprises a plurality of semiconductor layers each inherently designed to generate light at respective of a range of wavelength emission spectrums, said semiconductor layers arranged relative to one another wherein the light generated from each of said layers combines to directly generate and emit the artificial light at the predetermined wavelength emission spectrum and level of illuminance.

9. An artificial lighting system as claimed in claim 8 wherein the plurality of semiconductor layers is in the form of a grid of light emitting diodes each inherently designed to generate light at respective of distinct wavelength or colour spectrums corresponding to the range of wavelength emission spectrums.

10. An artificial lighting system as claimed in claim 9 wherein the grid of light emitting diodes combine to directly generate and emit said artificial light.

11. An artificial light source for emitting artificial light for controlling myopia in humans, said light source being a light-emitting diode type designed to directly generate and emit artificial light without filters:
i) at a predetermined wavelength emission spectrum substantially simulating the effect of sunlight, said wavelength spectrum being within a range of wavelengths detectable by an individual's eyes at a retinal level;
ii) at a predetermined level of illuminance measured at the eyes of at least 300 lux and up to around 1000 lux measured at or around desk height;
iii) the predetermined wavelength emission spectrum being a) higher in its proportion of wavelengths at 480 nm±5 nm relative to neighbouring wavelengths, and b) relatively lower in its proportion of high energy visible light of wavelengths at between 415 nm to 455 nm,
said artificial light when exposed to an individuals eyes for a predetermined exposure period of time of at least 180 minutes on average per day and at least 1,800 lux-hours per day being sufficient to trigger a neurological response in the retina of each of the eyes which is effective in contributing to a reduction in the onset or progression of myopia in the individual's eyes.

12. An artificial light source as claimed in claim 11 wherein the artificial light source of the light-emitting diode type comprises a plurality of semiconductor layers each designed to generate light at respective of a range of wavelength emission spectrums, said semiconductor layers arranged relative to one another wherein the light generated from each of said layers combines to directly generate and emit the artificial light at the predetermined wavelength emission spectrum and level of illuminance.

13. An artificial light source as claimed in claim 12 wherein the plurality of semiconductor layers is in the form of a grid of light emitting diodes each inherently designed to generate and emit light at respective of distinct wavelength or colour spectrums corresponding to the range of wavelength emission spectrums.

14. An artificial light source as claimed in claim 13 wherein the grid of light emitting diodes combine to directly generate and emit said artificial light.

15. A method of controlling myopia in humans, said method comprising the steps of:
(A) directly generating and emitting artificial light from an artificial light source without filters, said artificial light being emitted:
i) at a predetermined wavelength emission spectrum substantially simulating the effect of sunlight, said wavelength spectrum being within a range of wavelengths detectable by an individual's eyes at a retinal level;
ii) at a predetermined level of illuminance measured at the eyes of at least 300 lux and up to around 1,000 lux measured at desk height;
iii) the predetermined wavelength emission spectrum being a) higher in its proportion of wavelengths at 480 nm±5 nm relative to neighbouring wavelengths, and b) relatively lower in its proportion of high energy visible light of wavelengths at between 415 nm to 455 nm,
(B) exposing an individual's eyes to the artificial light for a predetermined exposure period of time of at least 180 minutes on average per day and at least 1,800 lux-hours per day, said artificial light at said predetermined exposure being sufficient to trigger a neurological response in the retina of each of the eyes which is effective in contributing to a reduction in the onset or progression of myopia in the individual's eyes.

16. A method as claimed in claim 15 wherein the step of exposing the individual's eyes to artificial light involves varying one or more characteristics of the artificial light at predetermined time intervals within the predetermined exposure period.

17. A method as claimed in claim 16 wherein said one or more characteristics of the artificial light include spectral power distribution, wavelength emission spectrum, CCT, level of illuminance or luminance, exposure period of time, and periodicity of these characteristics.

18. A method as claimed in claim 15 wherein the step of exposing the individual's eyes to artificial light involves substantially continuous exposure to said artificial light at ambient levels of illuminance which substantially simulates sunlight in its chromatic range of wavelengths.

19. Use of an artificial light source in the manufacture of an artificial lighting system for controlling myopia in humans, said lighting system as claimed in claim 1.
